(19) United States
(12) Patent Application Publication
(10) Pub. No.: US 2026/0166316 A1
(43) Pub. Date: Jun. 18, 2026

(54) DEEP BRAIN STIMULATION TO TREAT FOCAL EPILEPSY WITH MOTOR AND SENSORY MANIFESTATIONS

(71) Applicant: University of Pittsburgh - Of the Commonwealth System of Higher Education, Pittsburgh, PA (US)

(72) Inventors: Arianna Damiani, Pittsburgh, PA (US); Elvira Pirondini, Pittsburgh, PA (US); Erinn Grigsby, Pittsburgh, PA (US); Jonathan Chu-an Ho, Camas, WA (US); Jorge Gonzalez-Martinez, Pittsburgh, PA (US); Marco Capogrosso, Pittsburgh, PA (US)

(73) Assignee: University of Pittsburgh - Of the Commonwealth System of Higher Education, Pittsburgh, PA (US)

(21) Appl. No.: 19/127,381

(22) PCT Filed: Nov. 8, 2023
(86) PCT No.: PCT/US2023/079159

§ 371 Date: May 5, 2025

Related U.S. Application Data
(60) Provisional application No. 63/424,362, filed on Nov. 10, 2022.

(51) Int. Cl. A61N 1/36

(52) U.S. Cl. CPC A61N 1/36025; A61N 1/36017; A61N 1/36034; A61N 1/36064; A61N 1/36178

(57) ABSTRACT

Disclosed herein are methods for treating focal epilepsy in a subject by applying a therapeutically effective amount of an electrical stimulus to thalamic neurons, or to sub-thalamic neurons that project to the thalamic neurons, with one or more electrodes of a deep brain stimulator implanted in the thalamus or subthalamus of the subject. Application of the electrical stimulus reduces the seizure-induced motor or sensory impairment in the subject.

### CROSS REFERENCE TO RELATED APPLICATIONS

This application claims priority to U.S. Provisional Application No. 63/424,362, filed Nov. 10, 2023, which is incorporated by reference in its entirety.

### FIELD

The present disclosure relates to neurostimulation for the treatment of focal epilepsy, such as medically refractory focal epilepsy, in a subject.

### BACKGROUND

Epilepsy is one of the most common brain disorders, characterized by chronically recurrent seizures resulting from excessive electrical discharges from groups of neurons. Epilepsy affects about 50 million people worldwide. About 40% of epilepsy patients have medically refractory epilepsy, which cannot be controlled by non-invasive medical therapy such as anti-epilepsy drugs. Medically refractory epilepsy is a debilitating illness where individuals lose their independence, causing profound behavioral, psychological, social, financial and legal issues.

For medically refractory epilepsy patients with focal epilepsy, the current standard of care is to resect the epileptogenic zone, which is the minimal area of brain tissue responsible for generating the recurrent seizure activity. However, this treatment is generally only recommended if the cortical area containing the epileptogenic zone is amenable for safe removal. In patients with focal epilepsy involving highly eloquent cortical areas such as the motor cortex, the risk of devastating neurological consequences as motor deficits, minimizes resection options.

### SUMMARY

Provided herein are methods for treating focal epilepsy, including medically refractory focal epilepsy, in a subject. The methods comprise applying a therapeutically effective amount of an electrical stimulus to thalamic neurons, or to sub-thalamic area fibers that project to the thalamic neurons, with one or more electrodes of a deep brain stimulator implanted in the thalamus or subthalamus of the subject and controlled by a neurostimulator. The thalamic neurons project to a target location in the premotor, motor, or sensory cortex of the subject that comprises an epileptogenic zone for seizure-induced motor or sensory impairment due to the focal epilepsy in the subject. The electrical stimulus is applied in an amount sufficient to inhibit epileptiform activity in the epileptogenic zone and reduce the seizure-induced motor or sensory impairment. In several implementations the electrical stimulus comprises electrical pulses having a pulse frequency of at least 100 Hz, such as between 100 Hz and about 1000 Hz, or between about 150 Hz and about 200 Hz.

In some implementations, the electrical stimulus is applied to neurons in at least one motor and/or sensory area(s) of the ventral thalamus, such as the ventralis oralis anterior nucleus (VOA), ventralis oralis posterior nucleus (VOP), ventralis intermediate nucleus (VIM), and ventralis caudalis (VC).

In some implementations, the neurostimulator is activated to apply the electrical stimulus in response to feedback (such as epileptiform activity indicating the presence or onset of a seizure) in the subject from one or more electrodes for sensing neural activity at the target location in the subject. In some implementations, the neurostimulator is configured for activation by the subject in response to sensation of an aura of the focal epilepsy.

Provided herein are methods that include applying an electrical stimulus with one or more electrodes to the neurons and white matter fibers located in the thalamus or subthalamic area, in an amount sufficient reduce the seizure-induced motor or sensory impairment in the subject. In some implementations, the electrodes are configured to selectively apply the electrical stimulus to axons of the neurons one or more motor and/or sensory area(s) of the ventral thalamus. In particular implementations, the electrical stimulus includes electrical pulses with an amplitude of less than about 10 mA, pulse widths between about 100 μs and about 2 ms, and/or a pulse frequency between about 100 Hz and about 1000 Hz. In particular examples, the electrical pulses include charge-balanced pulses. In these and further implementations, the electrical stimulus may be a continuous electrical stimulus, or else may be a closed-loop electrical stimulus.

In methods according to some implementations herein, an electrical stimulus is applied by one or more electrodes controlled by a neurostimulator. In some implementations, the neurostimulator is externalized with respect to the human subject. In further implementations, the neurostimulator is implanted in the human subject. For example, in specific implementations, the neurostimulator is fully implanted in the brain of the subject. In further implementations, the neurostimulator is implanted in the chest of the subject. In still further implementations, the neurostimulator is implanted in the belly of the subject.

In some implementations, a neurostimulator utilized in methods herein is a current or voltage-controlled stimulator. In particular implementations, the neurostimulator controls the application of an electrical stimulus in a phasic manner. For example, the stimulation parameters of the neurostimulator may change over the course of a detected seizure. In some implementations herein, the neurostimulator comprises at least one multiple contact lead that is configured to produce orientation-selective axonal stimulation.

The foregoing and other objects, features, and advantages of the implementations will become more apparent from the following detailed description, which proceeds with reference to the accompanying figures.

### BRIEF DESCRIPTION OF THE FIGURES

FIG. 1. Cortical evoked potentials recorded from primary sensory cortex (S1) and primary motor cortex (M1) following stimulation of the ventralis oralis posterior nucleus (VOP) in the thalamus in a non-human primate. Clear responses to the thalamic stimulation localized in M1 were observed, showing a highly specific thalamocortical pathway.

FIG. 2. Electrocortigraphic activity of primary motor cortex M1, gamma oscillations of M1 and ventralis oralis posterior nucleus (VOP) activity during a spontaneous seizure in a non-human primate. The epileptiform activity is characterized by an increase of high-frequency oscillations that occur in the gamma band. Highlighted area shows a synchronized slow electrical potential drift in the VOP accompanied by an enhancement of high frequency activity power simultaneous with the occurrence of epileptiform cortical activity.

FIG. 3. Effect of VOP stimulation at different frequencies in a non-human rpimate. Left panel shows cortical activity and cortical gamma oscillations with various stimulation frequencies of the VOP. On the right panel, the RMS value of gamma oscillations is calculated.

FIG. 4. Effect of VOP stimulation on penicillin-induced epileptiform activity in a non-human primate. On the left, the raw trace shows interictal spikes during and after stimulation. On the right, the box plot represents the amplitude of IIS, which significantly decreases with stimulation (Bootstrap, alpha<0.05). On the right, the bar plot reports the reduction in IIS rate with stimulation.

FIGS. 5A and 5B. Involvement of the motor thalamus during seizures in a human patient with Rolandic epilepsy. (5A) Tractography of a human subject showing the fiber projections from VOP/VIM towards the motor and sensory cortex. (5B) On the top panel, cortical evoked potential elicited by VOP/VIM stimulation. On the bottom, heatmap representing the peak-to-peak amplitude of evoked potential recorded in various motor and sensory areas.

FIGS. 6A and 6B. Involvement of the motor thalamus during seizures in a human patient with Rolandic epilepsy. (FIG. 6A) Example of intracranial recording during a seizure in a human patient affected by Rolandic epilepsy. Top and bottom trace represent the cortical trace (seizure focus) and the thalamic trace, respectively. (FIG. 6B) h2 nonlinear correlation coefficient calculated between the motor thalamus and the seizure focus on the cortex. Left and right represent two contacts both located in the motor thalamus. In both cases, the motor thalamus presents high correlation with the seizure onset zone, specifically around the seizure termination.

FIGS. 7A and 7B. Example of the effect of electrical stimulation of the motor thalamus (FIG. 7A, 100 Hz; FIG. 7B, 50 Hz) in a human patient affected by Rolandic epilepsy. Quantitative analysis of the amplitude and rate of the interictal spikes (IIS) before, during, and after stimulation is also shown.

### DETAILED DESCRIPTION

### I. Introduction

Methods as disclosed herein arise from the unexpected discovery that deep brain stimulation (e.g., continuous stimulation) of specific areas in the ventral motor/sensory thalamus and subthalamic areas lead to suppression of epileptiform activity in an epileptogenic zone in a primate. As will be understood by those in the art, this finding has broad implications for the treatment of focal seizures in a subject.

Using available technology, therapeutic electrical stimuli may be administered to a subject by implanted electrodes under the control of an implanted or external neurostimulator. The electrodes and neurostimulator together comprise a system that may be provided to a subject as an assistive device to reduce epileptic symptoms, including onset, duration, and frequency of seizures with motor and sensory manifestations. This system may alternatively be used in combination with anti-epileptic drugs as known in the art for treatment of epilepsy.

As shown in the Examples, methods according to disclosed implementations reduce cortical activity and epileptiform activity in the epileptogenic zone and provide, an alternative to epileptogenic zone resection for treatment of focal epilepsy.

### II. Summary of Terms

Unless otherwise noted, technical terms are used according to conventional usage. As used herein, the term “comprises” means “includes.” Although many methods and materials similar or equivalent to those described herein can be used, particular suitable methods and materials are described below. In addition, the materials, methods, and examples are illustrative only and not intended to be limiting. The scope of the claims should not be limited to those features exemplified. To facilitate review of the various implementations, the following explanations of terms are provided:

About: As used herein, the term “about” refers to an approximation of a qualitative or quantitative measurement. Whether the measurement is qualitative or quantitative should be clear from its context. With regard to quantitative measurements, “about” refers to plus or minus 5% of a reference value. For example, “about” 100 mA refers to 95 mA to 105 mA.

Closed-loop: A stimulation mechanism wherein a sensor continuously records a feedback signal (for example, a signal correlated or causally linked to a motor deficit in a subject with a motor disorder), and a neurostimulator adjusts parameters of electrode control signals according to the feedback signal. Some implementations herein utilize such a closed-loop stimulation mechanism. In specific examples, specific frequency bands, recorded from an epileptogenic zone, trigger application of electrical stimulus via one or more electrodes to thalamic or subthalamic neurons (for example, in the ventral thalamus) comprising axons projecting to the epileptogenic zone. The sensor electrode can be located in the motor and sensory areas related to the epileptogenic zone (direct closed-loop system) or distant from the epileptogenic zone (remote closed-loop system).

Deep brain stimulation: Direct or indirect application of a stimulus to an area within the brain. In specific implementations herein, selective deep brain stimulation of neurons in somatotopically and/or stereotactically defined thalamic or subthalamic nuclei is accomplished via electrical stimulation. In alternative aspects, selective deep brain stimulation of neurons in somatotopically and/or stereotactically defined thalamic or subthalamic nuclei may be accomplished by optical stimulation via implanted optical fibers, magnetic stimulation, or pharmacological stimulation.

Electrical stimulus: The passing of various types of current or voltage selectively through one or more electrodes to a target location in a subject (for example, specific areas of the ventral thalamus).

Electrode: An electric conductor through which an electric current can pass. An electrode can also be a collector and/or emitter of an electric current. In some implementations, an electrode is a solid and comprises a conducting metal as the conductive layer. Non-limiting examples of conducting metals include noble metals and alloys, such as stainless steel and tungsten. An array of electrodes refers to a device with at least two electrodes formed in any pattern. A multi-channel electrode includes multiple conductive surfaces that can independently activated to stimulate or record electrical current.

Epilepsy: A brain disorder characterized by chronically recurrent seizures resulting from excessive electrical discharges from groups of neurons. Focal epilepsy is characterized by recurrent seizures arising from a specific region of the brain (e.g., the epileptogenic zone). A significant percentage of individuals with epilepsy have medically refractory epilepsy, which cannot completely be controlled by medical therapy, that is, seizures continue to occur despite treatment with a maximally tolerated dose of anti-epilepsy drugs.

Epileptogenic zone: The minimal area of brain tissue responsible for generating recurrent seizure activity in a patient with focal epilepsy. Various non-invasive and invasive methods are available for identification of an epileptogenic zone in a patient. Non-limiting examples of non-invasive techniques include monitoring of neural activity using scalp EEG, video-EEG, stereo-EEG, and brain imaging (e.g., MRI, PET, Ictal SPECT), as well as neuropsychological tests and speech-language studies. Non-limiting examples of invasive techniques include monitoring of neural activity using neural implants such as subdural grid and strip electrodes, stereotactically placed depth electrodes, which his typically performed in a dedicated Epilepsy Monitoring Unit (EMU)

Implanting: Completely or partially placing a neurodevice (such as a neurostimulator connected to an electrode array within a subject, for example, using surgical techniques. A neurodevice is partially implanted when some of the device or neurostimulator reaches, or extends to the outside of, a subject. A neurodevice can be implanted for varying durations, such as for a short-term duration (e.g., one or two days or less) or for long-term or chronic duration (e.g., one month or more).

Interictal spikes: interictal spikes (IIS) are large intermittent morphologically defined electrophysiological events observed between seizures in subjects with epilepsy. The spikes are generated by the synchronous discharges of a group of neurons in a region referred to as the epileptic focus. Interictal spikes are highly correlated with spontaneous seizures, and their presence is a factor that can be used to support the diagnosis of epilepsy.

Motor cortex and pre-motor cortex: The term “motor cortex” refers to an area within the cerebral cortex of the brain that is involved in the planning, control, and execution of voluntary movements. The motor cortex is situated within the frontal lobe of the brain, next to the central sulcus. The motor cortex is the only motor control center above the spinal cord that can directly communicate with most of the other motor control structures, such as the thalamus. The term “pre-motor cortex” refers to an area located just anterior to the primary motor cortex, which is involved in planning and organizing movements and actions. Neuronal activity in pre-motor cortex typically precedes activation of the primary motor cortex.

Motor threshold: The minimum thalamic or subthalamic stimulation intensity that can produce a motor output of a given amplitude from a muscle at rest (RMT) or during a muscle contraction (AMT).

Neural signal: An electrical signal originating in the nervous system of a subject. “Stimulating a neural signal” refers to application of an electrical current to the neural tissue of a subject in such a way as to cause neurons in the subject to produce an electrical signal (e.g., an action potential). An extracellular electrical signal can, however, originate in a cell, such as one or more neural cells. An extracellular electrical signal is contrasted with an intracellular electrical signal, which originates, and remains, in a cell. An extracellular electrical signal can comprise a collection of extracellular electrical signals generated by one or more cells.

Neurostimulator: A current or voltage-controlled electrical stimulation device. A neurostimulator controls the delivery of an electrical pulse, or pattern of electrical pulses, having defined parameters, for example and without limitation, pulse frequency, duration, amplitude, phase symmetry, duty cycle, pulse current, pulse width, and on-time and off-time. The controlled electrical pulse is delivered through one or more electrodes (for example, leadless electrode(s), or electrode(s) located at the end of a lead, a thin insulated wire) configured to apply the electrical stimulus to the brain of a subject. A neurostimulator may comprise at least one multiple contact lead. Neurostimulators may be utilized to apply a series of electrical pulse stimuli (e.g., charge balanced pulses) through at least one electrode; for example and without limitation, low-frequency pulse train patterns, frequency-sequenced pulse burst train patterns (e.g., wherein different sequences of modulated electrical stimuli are generated at different burst frequencies), and phasic train patterns (e.g., wherein the stimulus control parameters change over the course of feedback, from a distal source).

Perceptual threshold: The minimum thalamic or subthalamic stimulation intensity necessary for a conscious organism to be aware of a particular sensation.

Seizure: A period of symptoms due to abnormally excessive or synchronous neuronal activity in the brain. Generalized seizures affect both hemispheres of the brain. A focal seizure affects only one hemisphere. Symptoms of the seizure depend on the location of the abnormally excessive or synchronous neuronal activity, for example, seizure activity in the motor cortex may induce motor impairment in the patient.

Seizure-induced motor impairment: Acute partial or total loss of function of a body part, for example, limbs, hands, fingers, neck, tongue, mouth, and face muscles due to seizures resulting from excessive electrical discharges from groups of neurons. Particular motor impairments include loss of muscle strength, partial paralysis (paresis), loss of dexterity (such as hand finger movement), uncontrollable muscle tone, muscle twitching, jerking, spasms, repeated movements such as clawing or chewing).

Seizure-induced sensory impairment: Acute changes in sensation due to seizures resulting from excessive electrical discharges from groups of neurons. Particular sensory impairments include waves of hot or cold sensation, goosebumbs, tingling or numbness, auditory effects (e.g., buzzing sounds), and visual effects (e.g., flashing lights).

Sensory cortex: The term “sensory cortex” refers to an area within the cerebral cortex of the brain that is involved in the sensory manifestation processing. The sensory cortex is situated posterior to the central sulcus, in the parietal lobe.

Subject: Living multi-cellular vertebrate organisms, a category that includes human and non-human mammals, including non-human primates, rats, mice, guinea pigs, cats, dogs, cows, horses, and the like. Thus, the term “subject” includes both human and veterinary subjects.

Subthalamic area: A region of several grey matter nuclei and surrounding white matter structures located ventral to the thalamus, medial to the internal capsule and lateral to the hypothalamus. Subthalamic structures include the subthalamic nucleus, the zona incerta, the ansa lenticularis, and the Fields of Forel. The Field H₁ of Forel (also known as the thalamic fascicle) is a horizontal white matter tract composed of the ansa lenticularis, lenticular fasciculus, and cerebellothalamic tracts between the subthalamus and the thalamus. These fibers are projections to the ventral anterior and ventral lateral thalamus from the basal ganglia and the cerebellum.

Therapeutically effective amount: An amount sufficient to provide a beneficial, or therapeutic, effect to a subject or a given percentage of subjects. Therapeutically effective amounts of a treatment can be determined in many different ways, such as assaying for a reduction in a disease or condition (such as motor or sensory impairment to due epileptic seizure). Therapeutic treatments can be administered in a single application, or in several applications (e.g., chronically over an appropriate period of time). However, the effective amount can be dependent on the source applied, the subject being treated, the severity and type of the condition being treated, and the manner of administration.

Treating or treatment: With respect to disease or condition (e.g., motor or sensory impairment due to focal epilepsy), either term includes (1) preventing the disease or condition, e.g., causing the clinical symptoms of the disease or condition not to develop in a subject that may be exposed to or predisposed to the disease or condition but does not yet experience or display symptoms of the disease or condition, (2) inhibiting the disease or condition, e.g., arresting the development of the disease or condition or its clinical symptoms, or (3) relieving the disease or condition, e.g., causing regression of the disease or condition or its clinical symptoms.

Thalamus: A paired structure of gray matter located in the forebrain with nerve fibers projecting to multiple brain structure, including the hippocampus and cerebral cortex. The thalamus is divided into several sections, including the median, medial, anterior, and ventral thalamus, which contain different nuclei projecting to defined cortical regions.

Ventral thalamus: An area of the thalamus comprising the reticular nucleus, the zona incerta, and the ventral lateral geniculate nucleus. As used herein, “ventral thalamus” may refer to a set of particular thalamic nuclei including, for example and without limitation, ventral thalamic nuclei comprising primary thalamic relays for motor and sensory information from the body and head (e.g., the ventralis anterior nucleus (VA), the ventralis oralis posterior nucleus (VOP), the ventralis intermediate nucleus (VIM), the ventralis caudal nucleus (VC), lateral areas of the VOP and/or VIM (e.g., a lateral area of the VOP and/or VIM associated with arm movements), and medial areas of the VOP associated with face movements). Certain motor and sensory thalamic nuclei herein may be stereotactically defined by reference to one or more the following AC-PC-based stereotactic coordinates: lateral (from about 5 to about 16 mm lateral to the AC/PC line); anterior/posterior (from about 2 to about 10 mm anterior to PC); and dorsal/ventral (from about +2 to about −6 mm from the AC/PC plane.

### III. Deep Brain Stimulation of Specific Ventral Thalamic Nuclei to Treat Intractable Seizures With Motor and Sensory Manifestations

Provided herein are methods for treating a subject (for example, a human subject) having focal epilepsy, such as medically refractory focal epilepsy, causing motor or sensory impairment. Methods according to particular implementations disclosed herein may be utilized to treat (i.e., prevent, ameliorate, suppress, and/or alleviate) a subject's seizure-induced motor or sensory impairment. In particular implementations herein, a disclosed method is utilized to treat a subject with Rolandic epilepsy (including Rolandic epilepsy with motor and/or sensory manifestations).

In some implementations, a subject with focal epilepsy, such as medically refractory focal epilepsy, is selected for treatment, and the method comprises implanting the deep brain stimulator in the subject.

The provided methods include applying a therapeutically effective amount of an electrical stimulus to thalamic neurons, or to sub-thalamic neurons that project to the thalamic neurons, with one or more electrodes of a deep brain stimulator implanted in the thalamus or subthalamus of the subject and controlled by a neurostimulator. The thalamic neurons project to a target location in the premotor, motor, or sensory cortex of the subject that comprises an epileptogenic zone for seizure-induced motor or sensory impairment due to the focal epilepsy in the subject. The electrical stimulus is applied in an amount sufficient to reduce the seizure-induced motor or sensory impairment in the subject.

In some implementations, a method for treating the subject having focal epilepsy, such as medically refractory focal epilepsy, causing seizures with motor impairment comprises applying a therapeutically effective amount of a stimulus (e.g., an electrical stimulus) to neurons in the thalamus, wherein the neurons comprise axons projecting to premotor or motor cortex. The electrical stimulus can be applied with one or more electrodes controlled by a neurostimulator. The electrical stimulus reduces the seizure-induced motor impairment in the subject, for example, by reducing the frequency or severity of the seizures. In some aspects, applying the therapeutically effective amount of the stimulus reduces the frequency or severity of the seizures in the subject by at least 50%, such as by at least 75% or elimination of the seizures or the motor impairment induced by the seizures.

In some implementations, a method for treating the subject having focal epilepsy, such as medically refractory focal epilepsy, causing seizures with sensory impairment comprises applying a therapeutically effective amount of a stimulus (e.g., an electrical stimulus) to neurons in the thalamus, wherein the neurons comprise axons projecting to sensory cortex. The electrical stimulus can be applied with one or more electrodes controlled by a neurostimulator. The electrical stimulus reduces the seizure-induced sensory impairment in the subject, for example, by reducing the frequency or severity of the seizures. In some aspects, applying the therapeutically effective amount of the stimulus reduces the frequency or severity of the seizures in the subject by at least 50%, such as by at least 75% or elimination of the seizures or the sensory impairment induced by the seizures.

In some implementations, a method for treating the subject having focal epilepsy, such as medically refractory focal epilepsy, causing seizures with motor impairment comprises applying a therapeutically effective amount of a stimulus (e.g., an electrical stimulus) to neurons in the subthalamus, wherein the neurons comprise axons projecting to neurons of the thalamus that project to premotor or motor cortex. The electrical stimulus can be applied with one or more electrodes controlled by a neurostimulator. The electrical stimulus reduces the seizure-induced motor impairment in the subject, for example, by reducing the frequency or severity of the seizures. In some aspects, applying the therapeutically effective amount of the stimulus reduces the frequency or severity of the seizures in the subject by at least 50%, such as by at least 75% or elimination of the seizures or the motor impairment induced by the seizures.

In some implementations, a method for treating the subject having focal epilepsy, such as medically refractory focal epilepsy, causing seizures with motor impairment comprises applying a therapeutically effective amount of a stimulus (e.g., an electrical stimulus) to neurons in the subthalamus, wherein the neurons comprise axons projecting to neurons of the thalamus that project to sensory cortex. The electrical stimulus can be applied with one or more electrodes controlled by a neurostimulator. The electrical stimulus reduces the seizure-induced motor impairment in the subject, for example, by reducing the frequency or severity of the seizures. In some aspects, applying the therapeutically effective amount of the stimulus reduces the frequency or severity of the seizures in the subject by at least 50%, such as by at least 75% or elimination of the seizures or the sensory impairment induced by the seizures.

Some implementations of methods disclosed herein include the specific stimulation of one or more ventral nuclei of the thalamus, for example, one or more motor and sensory areas of the thalamus (e.g., ventral anterior nucleus (VA), the ventralis oralis posterior nucleus (VOP), the ventralis oralis anterior nucleus (VOA), or the ventralis intermediate nucleus (VIM), ventralis caudalis (VC)). In particular implementations, stimulation of specific areas of the ventral thalamus targets fibers connecting the thalamus to the pre-motor and motor cortices to an extent sufficient to suppress epileptiform activity in the epileptogenic zone. In some implementations, the electrical stimulus is not applied to the anterior or centromedian nucleus of the thalamus.

Some implementations of methods disclosed herein include the specific stimulation of one or more ventral nuclei of the thalamus, for example, one or more motor and sensory areas of the thalamus (e.g., ventral anterior nucleus (VA), the ventralis oralis posterior nucleus (VOP), the ventralis oralis anterior nucleus (VOA), or the ventralis intermediate nucleus (VIM), ventralis caudalis (VC)). In particular implementations, stimulation of specific areas of the ventral thalamus targets fibers connecting the thalamus to the pre-motor and motor cortices to an extent sufficient to suppress epileptiform activity in the epileptogenic zone. In some implementations, the deep brain stimulator is implanted to target neurons within a region of the thalamus defined by AC/PC stereotactic coordinates, such as: Anterior-Posterior: 4-10 mm from PC; Mesial-Lateral: 12-16mm from the AC/PC line; and Dorsal-Ventral: +2 to −6 mm from the AC/PC plane.

Some implementations of methods disclosed herein include the specific stimulation of an subthalamus area, for example, white matter fibers in the Field H₁ of Forel. In particular implementations, stimulation of specific areas of the subthalamus targets fibers projecting to areas of the thalamus containing neurons that project to the pre-motor and motor cortices to an extent sufficient to suppress epileptiform activity in the epileptogenic zone.

In some implementations, the electrical stimulus includes electrical pulses defined by parameters including, for example and without limitation, amplitude, pulse width, and pulse frequency. Such electrical pulses may include charge-balanced pulses. In these and further implementations, the electrical stimulus may be a continuous electrical stimulus, and/or a closed-loop electrical stimulus.

In particular examples, the electrical stimulus includes electrical pulses with an amplitude of less than about 10 mA; for example, less than 10 mA, less than 9 mA, less than 8 mA, less than 7 mA, less than 6 mA, less than 5 mA, less than 4 mA, less than 3 mA, less than 2 mA, or less than 1 mA. In particular examples, the electrical stimulus includes electrical pulses with pulse widths between about 80 μs and about 2 ms; for example, between 80 μs and 2 ms, between 100 μs and 2 ms, between 200 μs and 2 ms, between 300 μs and 2 ms, between 400 μs and 2 ms, between 500 μs and 2 ms, between 600 μs and 2 ms, between 700 μs and 2 ms, between 800 μs and 2 ms, between 800 μs and 2 ms, between 900 μs and 2 ms, between 1 ms and 2 ms, between 1.5 ms and 2 ms, between 80 μs and 1.5 ms, between 100 μs and 1.5 ms, between 200 μs and 1.5 ms, between 300 μs and 1.5 ms, between 400 μs and 1.5 ms, between 500 μs and 1.5 ms, between 600 μs and 1.5 ms, between 700 μs and 1.5 ms, between 800 μs and 1.5 ms, between 800 μs and 1.5 ms, between 900 μs and 1.5 ms, between 1 ms and 1.5 ms, between 1.5 ms and 2 ms, between 80 μs and 1 ms, between 100 μs and 1 ms, between 200 μs and 1 ms, between 300 μs and 1 ms, between 400 μs and 1 ms, between 500 μs and 1 ms, between 600 μs and 1 ms, between 700 μs and 1 ms, between 800 μs and 1 ms, between 800 μs and 1 ms, and between 900 μs and 1 ms. In particular examples, the electrical stimulus includes a pulse frequency between about 100 Hz and about 1000 Hz; for example, between 50 Hz and 1000 Hz, between 100 Hz and 1000 Hz, between 100 Hz and 900 Hz, between 100 Hz and 800 Hz, between 100 Hz and 700 Hz, between 100 Hz and 600 Hz, between 100 Hz and 500 Hz, between 100 Hz and 400 Hz, between 100 Hz and 300 Hz, and between 100 Hz and 200 Hz. In some implementations, the pulse frequency is between 100 Hz and 250 Hz.

Accordingly, the electrical stimulus in particular implementations herein includes electrical pulses having an amplitude of less than about 10 mA, a pulse width of between about 100 μs and about 2 ms, and a pulse frequency between about 100 Hz and about 1000 Hz, such as between about 100 Hz and about 250 Hz or between about 150 Hz and about 200 Hz.

In particular implementations, the stimulation is applied at or below the subject's motor threshold and/or at or below the subject's perceptual threshold. For example, the stimulation may be applied below the motor threshold and below the perceptual threshold; below the motor threshold, but at or above the perceptual threshold; or below the perceptual threshold, but at or above the motor threshold.

In some implementations, disclosed methods are effected by the use of an implanted neurostimulator that controls the stimulation (e.g., electrical stimulation via one or more implanted electrode(s)) according to predetermined parameters or parameters determined by feedback in a closed-loop system. In particular implementations, methods disclosed herein can be used in combination with motor rehabilitation therapy to improve long-term recovery outcomes.

Deep brain stimulation is an advanced neurosurgical procedure involving the implantation of one or more electrode(s) that deliver an electrical stimulus under the control of an externalized or implanted neurostimulator unit. Implantation of the electrode(s), and/or a neurostimulator in examples where the neurostimulator is not externalized, is typically performed by a clinical team including neurologists, neurosurgeons, neurophysiologists, and other specialists trained in the assessment, treatment, and care of neurological conditions. Typically, following selection of an appropriate subject and determination of the area of the subject's brain to be stimulated, precise placement of at least one electrode in the area of the patient's thalamus or subthalamus is carried out in an operating room setting, typically utilizing brain imaging technology and stereotactic targeting made possible by the stereotypical organization of different areas of the thalamus or subthalamus. After administration of local anesthesia, the subject undergoing electrode implantation experiences little discomfort, and is generally kept awake during the implantation procedure to allow communication with the surgical team.

Some implementations herein employ an implant that includes one or more electrodes and/or neurostimulator implanted (e.g., fully or partially implanted) in the brain of a subject. Further implementations herein employ an implant that includes one or more magnets or optical fibers, and/or a neurostimulator implanted in the brain of a subject.

Numerous types and styles of neural implants (for example, implants including one or more electrodes for providing an electrical stimulus) are available and known to those in the art. Any neural implant for specific stimulation of a thalamic or subthalamic area in a subject may be utilized in specific implementations. In some implementations, more than one electrode is implanted, such as an array of electrodes. In additional implementations, a device is provided that can include one or more electrodes. Non-limiting examples include deep brain stimulators, EcoG grids, electrode arrays, microarrays (e.g., Utah and Michigan microarrays), and microwire electrodes and arrays.

In some implementations, an implanted neurostimulator can be used for stimulating bio-electric (e.g., neural) signals to thalamic and subthalamic area in the subject. For example, an implanted neurostimulator may be implanted so as to specifically stimulate one or more area of a subject's ventral thalamus for a period of at least 1 month; for example, at least 2, 6, 12, 18, 24, 30, 36, or more months.

In some implementations, circuitry is implanted connecting a neurostimulator to the one or more electrodes. In particular implementations, the circuits are fully implanted (typically in a subcutaneous pocket within a subject's body), or are partially implanted in the subject. The operable linkage of the neurostimulator to the electrode(s) can be by way of one or more leads, although any operable linkage capable of transmitting a stimulation signal from the circuitry to the electrodes may be used in specific implementations.

In some implementations, electrodes used in accordance with the invention are positioned in specific areas of the brain (such as the ventral thalamus), so as to be capable of selective application of an electrical stimulus to the specific area, by any of the methods conventionally used for positioning of electrodes for deep brain stimulation. As is known in the art, the particular procedures used will vary according to the available equipment, training of personnel, and the circumstances of each case. Detailed examples of such procedures are described, for example, in Benabid et al., Movement Disorders 17 (Suppl. 3): S123-129 (2002), and in Schrader et al., Movement Disorders 17 (Suppl. 3): S167-174 (2002). In some examples, the procedures for placement and testing of electrodes are divided into several steps including mounting of a stereotactic ring on the patient's skull, and imaging by high resolution stereotactic commuted tomographic (CT) scanning of the head. The stereotactic CT scan is preferably preceded by high resolution, volumetric, and three tesla magnetic resonance imaging (MRI) in advance of placement of the stereotactic head ring. Planning of the surgical target sites within the brain and trajectories for approach to the selected targets can be achieved using the MRI images and computer software designed for stereotactic targeting, for example, Stereoplan™ Plus 2.3 (Stryker-Leibinger, Friedburg, Germany), and SNS™ 3.14 (Surgical Navigation Specialists, Mississauga, Canada).

Post-operative control of selective electrical stimulation of the thalamic and subthalamic areas by the implanted electrode is provided in some implementations by a neurostimulator that may be externalized or implanted; for example, subcutaneously (e.g., in the chest or belly of the subject). Following recovery from the implantation, surgery, and connection of electrode leads to the neurostimulator, the subject may be monitored and tested to establish parameters for the electrical stimulation based on the subject's condition. In some implementations, electrical stimulation by the implanted electrode(s) is delivered to at least one specific area of the subject's ventral thalamus while the subject is monitored for seizure activity. In some implementations, the parameters of the electrical stimulus controlled by the neurostimulator are adjusted according to changes in the seizure activity due to the applied stimulus, for example, so as to reduce the frequency or severity of seizures with minimal side effects due to the applied electrical stimulus. In specific implementations, the operation of the device and/or the neurostimulator can be at least partially under the control of the subject once the subject is released from a clinical setting. For example, the subject can activate the neurostimulator in response to sensation of an aura of the focal epilepsy. In these and further implementations, the subject is taught how to use the device and/or the neurostimulator.

### Additional Description

Clause 1. A method for treating medically refractory focal epilepsy in a subject, comprising:
applying a therapeutically effective amount of an electrical stimulus to thalamic neurons, or to sub-thalamic white matter fibers that project to the thalamic neurons, with one or more electrodes of a deep brain stimulator implanted in the thalamus or subthalamus of the subject and controlled by a neurostimulator, and wherein:
   the thalamic neurons project to a target location in the premotor, motor, or sensory cortex of the subject;
   the target location comprises an epileptogenic zone for seizure-induced motor or sensory impairment due to the medically refractory focal epilepsy in the subject; and
   the electrical stimulus is applied in an amount sufficient to reduce the seizure-induced motor or sensory impairment in the subject.

Clause 2. The method of clause 1, wherein applying the therapeutically effective amount of the electrical stimulus inhibits epileptiform activity in the epileptogenic zone.

Clause 3. The method of any one of the prior clauses, wherein the electrical stimulus comprises electrical pulses having a pulse frequency of at between 100 Hz and about 1000 Hz.

Clause 4. The method of any one of the prior clauses, wherein the electrical stimulus comprises electrical pulses having an amplitude of less than about 10 mA, pulse widths between about 100 μs and about 2 ms, and/or a pulse frequency between about 100 Hz and about 1000 Hz.

Clause 5. The method of any one of the prior clauses, wherein the electrical stimulus comprises electrical pulses having a pulse frequency of between 100 Hz and 250 Hz.

Clause 6. The method of clauses 1-4, wherein the electrical stimulus comprises electrical pulses having a pulse frequency of between 150 Hz and 200 Hz.

Clause 7. The method of any one of the prior clauses, wherein the electrical stimulus is applied to neurons in at least one motor and/or sensory area(s) of the ventral thalamus.

Clause 8. The method of clause 7, wherein the electrical stimulus is applied to the ventral anterior nucleus (VA), the ventral oralis posterior nucleus (VOP), the ventral oralis anterior nucleus (VOA), the ventralis intermediate nucleus (VIM), the ventralis caudalis (VC).

Clause 9. The method of any one of the prior clauses, wherein the thalamic neurons are located with a region of the thalamus defined by the following AC/PC stereotactic coordinates: Anterior-Posterior: 4-10 mm from PC; Mesial-Lateral: 12-16 mm from the AC/PC line; and Dorsal-Ventral: +2 to −6 mm from the AC/PC plane.

Clause 10. The method of any one of clauses 1-6, wherein the electrical stimulus is applied to the white matter fibers in the Field H₁ of Forel of the sub-thalamic area.

Clause 11. The method of any one of the prior clauses, wherein the neurostimulator is activated to apply the electrical stimulus in response to feedback from one or more electrodes for sensing neural activity at the target location in the subject.

Clause 12. The method of clause 11, wherein the feedback is epileptiform activity indicating the presence or onset of a seizure in the subject.

Clause 13. The method of any one of the prior clauses, wherein the neurostimulator is configured for activation by the subject in response to sensation of an aura of the medically refractory focal epilepsy.

Clause 14. The method of any one of the prior clauses, wherein the neurostimulator is an external or implanted pulse generator.

Clause 15. The method of any one of the prior clauses, wherein the neurostimulator delivers the electrical stimulus as charge balanced pulses, a continuous electrical stimulus, and/or a closed-loop electrical stimulus.

Clause 16. The method of any one of the prior clauses, wherein the target location is an epileptogenic zone for seizure-induced motor impairment in the subject and the electrical stimulus is applied at or below a motor threshold such that the electrical stimulus does not directly elicit movement in the subject.

Clause 17. The method of any one of the prior clauses, wherein:
the medically refractory focal epilepsy is Rolandic epilepsy, the electrodes of the deep brain stimulator is implanted at the motor thalamus, and the electrical stimulus comprises electrical pulses having a pulse frequency of between 100 Hz and 250 Hz.

Clause 18. The method of any one of the prior clauses, further comprising selecting the subject with the medically refractory focal epilepsy for treatment and implanting the deep brain stimulator in the subject.

Clause 19. The method of any one of the prior clauses, wherein the electrical stimulus is not applied to the anterior or centromedian nucleus of the thalamus.

### Additional Description

Aspect 1. A method for treating focal epilepsy in a subject, comprising:
applying a therapeutically effective amount of an electrical stimulus to thalamic neurons, or to sub-thalamic white matter fibers that project to the thalamic neurons, with one or more electrodes of a deep brain stimulator implanted in the thalamus or subthalamus of the subject and controlled by a neurostimulator, and wherein:
   the thalamic neurons project to a target location in the premotor, motor, or sensory cortex of the subject;
   the target location comprises an epileptogenic zone for seizure-induced motor or sensory impairment due to the focal epilepsy in the subject; and
   the electrical stimulus is applied in an amount sufficient to reduce the seizure-induced motor or sensory impairment in the subject.

Aspect 2. The method of aspect 1, wherein the focal epilepsy is medically refractory focal epilepsy.

Aspect 3. The method of aspect 1 or aspect 2, wherein applying the therapeutically effective amount of the electrical stimulus inhibits epileptiform activity in the epileptogenic zone.

Aspect 4. The method of any one of the prior aspects, wherein the electrical stimulus comprises electrical pulses having a pulse frequency of between about 50 Hz and about 1000 Hz.

Aspect 5. The method of any one of the prior aspects, wherein the electrical stimulus comprises electrical pulses having an amplitude of less than about 10 mA, pulse widths between about 100 μs and about 2 ms, and/or a pulse frequency between about 100 Hz and about 1000 Hz.

Aspect 6. The method of any one of the prior aspects, wherein the electrical stimulus comprises electrical pulses having a pulse frequency of between about 100 Hz and about 250 Hz.

Aspect 7. The method of any one of aspects 1-6, wherein the electrical stimulus comprises electrical pulses having a pulse frequency of between about 150 Hz and about 200 Hz.

Aspect 8. The method of any one of aspects 1-6, wherein the electrical stimulus comprises electrical pulses having a pulse frequency of about 100 Hz.

Aspect 9. The method of any one of the prior aspects, wherein the electrical stimulus is applied to neurons in at least one motor and/or sensory area(s) of the ventral thalamus.

Aspect 10. The method of aspect 9, wherein the electrical stimulus is applied to the ventral anterior nucleus (VA), the ventral oralis posterior nucleus (VOP), the ventral oralis anterior nucleus (VOA), the ventralis intermediate nucleus (VIM), the ventralis caudalis (VC).

Aspect 11. The method of aspect 10, wherein the electrical stimulus is applied to the ventralis intermediate nucleus (VIM).

Aspect 12. The method of aspect 10, wherein the electrical stimulus is applied to the ventral oralis posterior nucleus (VOP).

Aspect 13. The method of any one of the prior aspects, wherein the thalamic neurons are located with a region of the thalamus defined by the following AC/PC stereotactic coordinates: Anterior-Posterior: 4-10 mm from PC; Mesial-Lateral: 12-16 mm from the AC/PC line; and Dorsal-Ventral: +2 to −6 mm from the AC/PC plane.

Aspect 14. The method of any one of aspects 1-8, wherein the electrical stimulus is applied to the white matter fibers in the Field HI of Forel of the sub-thalamic area.

Aspect 15. The method of any one of the prior aspects, wherein the neurostimulator is activated to apply the electrical stimulus in response to feedback from one or more electrodes for sensing neural activity at the target location in the subject.

Aspect 16. The method of aspect 15, wherein the feedback is epileptiform activity indicating the presence or onset of a seizure in the subject.

Aspect 17. The method of any one of the prior aspects, wherein the neurostimulator is configured for activation by the subject in response to sensation of an aura of the focal epilepsy.

Aspect 18. The method of any one of the prior aspects, wherein the neurostimulator is an external or implanted pulse generator.

Aspect 19. The method of any one of the prior aspects, wherein the neurostimulator delivers the electrical stimulus as charge balanced pulses, a continuous electrical stimulus, and/or a closed-loop electrical stimulus.

Aspect 20. The method of any one of the prior aspects, wherein the target location is an epileptogenic zone for seizure-induced motor impairment in the subject and the electrical stimulus is applied at or below a motor threshold such that the electrical stimulus does not directly elicit movement in the subject.

Aspect 21. The method of any one of the prior aspects, wherein:
the focal epilepsy is Rolandic epilepsy, the electrodes of the deep brain stimulator are implanted at the motor thalamus, and the electrical stimulus comprises electrical pulses having a pulse frequency of between 100 Hz and 250 Hz.

Aspect 22. The method of any one of the prior aspects, further comprising selecting the subject with the focal epilepsy for treatment and implanting the deep brain stimulator in the subject.

Aspect 23. The method of any one of the prior aspects, wherein the electrical stimulus is not applied to the anterior or centromedian nucleus of the thalamus.

### EXAMPLES

The following examples are provided to illustrate particular features of certain implementations, but the scope of the claims should not be limited to those features exemplified.

### Example 1

### Deep Brain Ventral Thalamic Stimulation for Seizure Suppression in NHP

This example illustrates neurostimulation of the ventral oralis posterior nucleus (VOP) nucleus in the ventral thalamus to suppress epileptiform activity in the epileptogenic zone of the motor cortex in a non-human primate with chronic spontaneous epilepsy characterized by focal clonic motor seizures.

The current standard of care for medically refractory epilepsy relies on the focal resection of the epileptogenic zone if the cortical area is amenable for safe removal. In patients with focal epilepsy involving highly eloquent cortical areas such as the motor cortex, the risk of devastating neurological consequences as motor deficits, minimizes resection options.

Current understanding of specific changes in thalamocortical communication during focal motor seizures is inadequate. Evidence provided in this example characterizes the dynamic relation between thalamic nuclei and seizure activity in the motor cortex, and identifies targets of thalamic deep brain stimulation for focal medically refractory epilepsy.

Experiments were performed in acute preparations of anesthetized nonhuman primates (n=2). A depth electrode was implanted in the VOP using MRI-guided stereotaxic surgery, and a 96 intracortical array was implanted in the motor cortex. This allowed simultaneous stimulation of the thalamus and recording from motor cortex (M1). Stimulation was applied to the thalamus at frequencies varying from 1 to 200 Hz. Stimulation was applied to the thalamus at frequencies varying from 1 to 200 Hz. Pulse width was set to 300 μs and amplitude was set to 4 mA.

The second animal expressed chronic spontaneous epilepsy characterized by focal clonic motor seizures in the right forelimb.

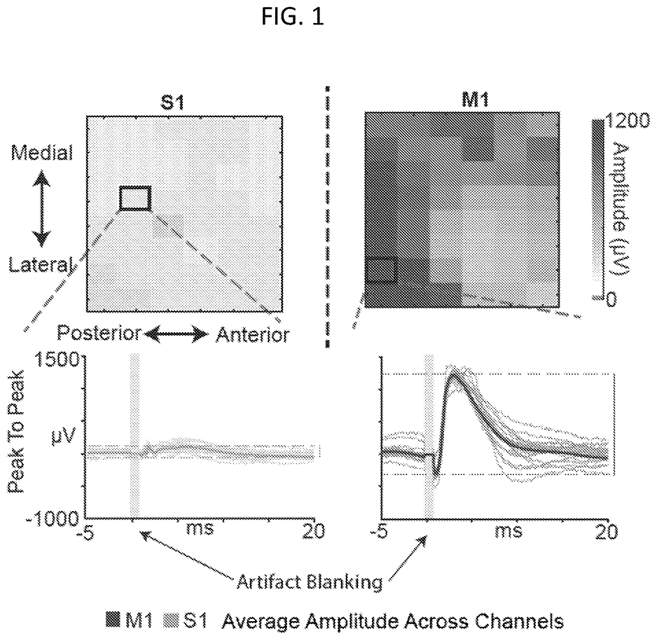

Following low-frequency VOP stimulation, we identified clear evoked potentials in ipsilateral M1, reflecting orthodromic monosynaptic neurotransmission (FIG. 1).

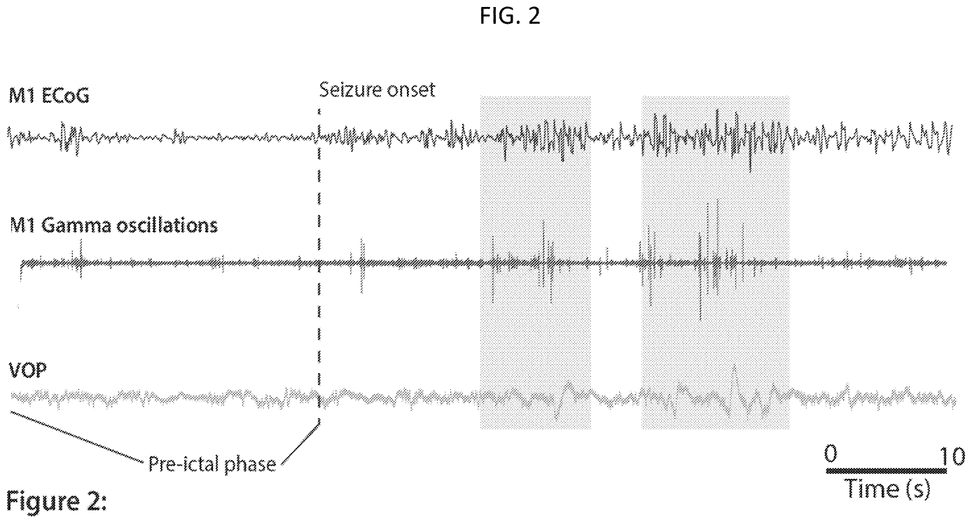

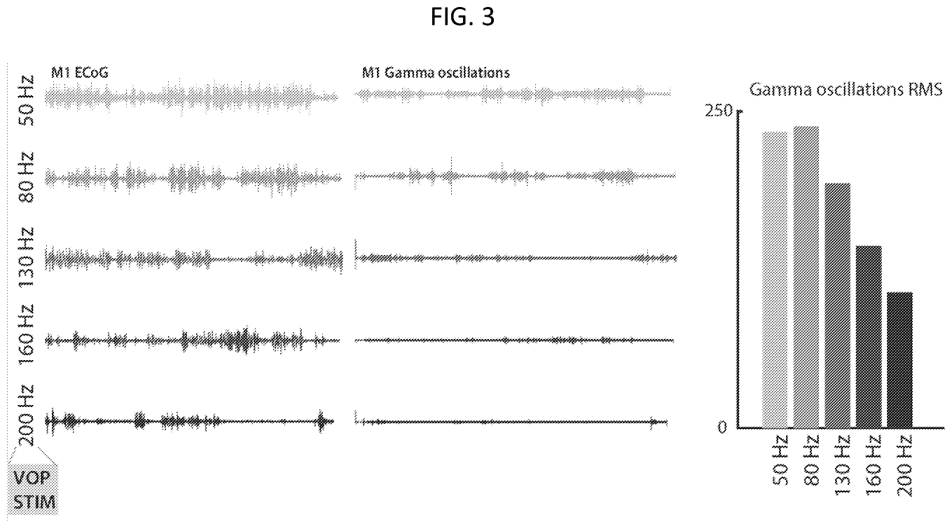

In the epileptic monkey, the intraoperative electrocorticography revealed the presence of repetitive polispikes located in the left motor and pre-motor cortex (FIG. 2). Interestingly, we observed synchronous thalamocortical connectivity enhanced during the ictal phase. The thalamic recording showed a slow potential drift accompanied by an enhancement of high frequency activity power simultaneous with the occurrence of epileptiform cortical activity. Importantly, we observed a frequency-dependent suppression of high-frequency cortical discharges with VOP stimulation (FIG. 3). Attenuation of the epileptic M1 activity started after 50 Hz stimulation, reaching an almost complete suppression at 200 Hz.

The results provide evidence of the electrophysiological interaction among cortical and subcortical areas, and demonstrate the suppression of epileptiform activity with high-frequency stimulation of the motor thalamus.

### Example 2

### Deep Brain Ventral Thalamic Stimulation for Seizure Suppression in NHP

This example illustrates neurostimulation of the ventral oralis posterior nucleus (VOP) in the ventral thalamus to suppress epileptiform activity in the epileptogenic zone of the motor cortex in a non-human primate with drug-induced epilepsy characterized by focal interictal spikes.

Evidence provided in this example identifies targets of thalamic deep brain stimulation for focal epilepsy, such as medically refractory focal epilepsy.

The experiment was in acute preparations of anesthetized nonhuman primate (n=1). A depth electrode was implanted in the VOP using MRI-guided stereotaxic surgery, and a 96 intracortical array was implanted in the motor cortex. This allowed simultaneous stimulation of the thalamus and recording from motor cortex (M1). Stimulation was applied to the thalamus at frequencies varying from 1 to 200 HzPulse width was set to 300 μs and amplitude was set to 1.2 mA.

The animal developed drug-induced epilepsy after the injection of 2500 IU of penicillin (G-sodium salt, 2%) in the motor cortex. This pathological model is widely used in the epilepsy field due to its action on inhibitory pathway inhibition that leads to increased neuronal excitability and lowered seizure threshold.

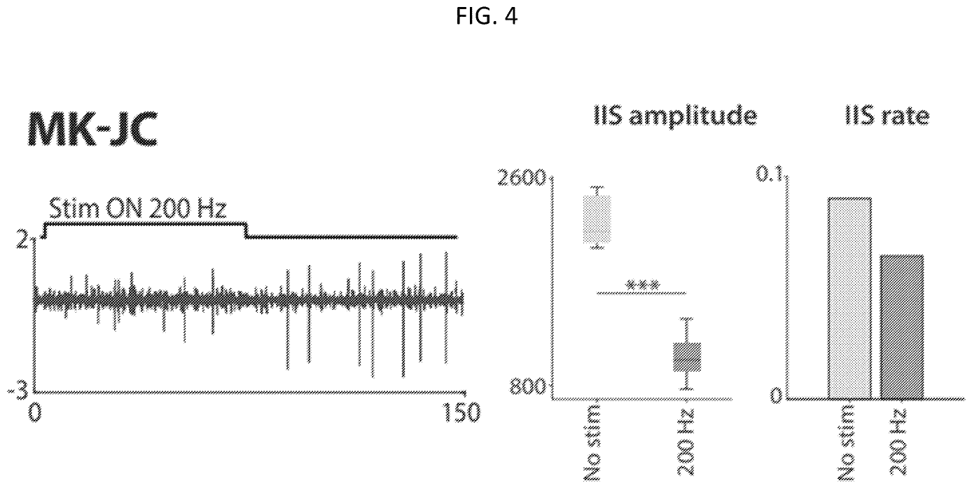

The intraoperative electrocorticography revealed the presence of repetitive interictal spikes located in the motor cortex. Following high-frequency stimulation of the VOP, we observed a significant reduction of the rate and amplitude of IIS (FIG. 4). The results demonstrate the suppression of epileptiform activity with high-frequency stimulation of the motor thalamus.

### Example 3

### Deep Brain Ventral Thalamic Stimulation for Seizure Suppression in Human

This example illustrates neurostimulation of the ventral oralis posterior nucleus (VOP) nucleus in the ventral thalamus to suppress epileptiform activity in the epileptogenic zone of the motor system (i.e., motor cingulate) in a human subject affected by Rolandic epilepsy.

Evidence provided in this example identifies targets of thalamic deep brain stimulation for focal epilepsy, such as medically refractory focal epilepsy, in humans.

### Methods

We performed electrophysiological experiments on a male patient. The patient was admitted to the Epilepsy Monitoring Unit (EMU) of the University of Pittsburgh Medical Center for a bilateral stereoelectroencephalography (SEEG) implantation to map his epileptogenic zone. During his stay in the EMU, we continuously recorded brain activity from 15 SEEG electrodes through the NATUS™ Medical system. The epileptogenic zone for this patient was defined as a focal area in the motor cingulate (electrodes Y1-4). Additionally, one of the SEEG electrode trajectories passed through the motor thalamus (VIM nucleus), allowing us to electrically stimulate from this region. This allowed simultaneous stimulation of the thalamus and recording from multiple areas of the brain, including the motor cingulate (epileptogenic zone), motor cortex, sensory cortex.

Ictal analysis: While the patient was monitored in the EMU, we recorded 59 spontaneous ictal events (i.e., seizures). For each of these, we analyzed correlation between bipolar signals locally recorded from different regions of interest, the motor thalamus and the epileptogenic zone. In order to quantify the interaction between the selected regions and to compare them with background activity, we chose three periods of interest: (i) background activity: including 10 s to 2 min before the onset of an ictal discharge and was used as a reference period; (ii) seizure onset: including the first 10 s after the beginning of a low-voltage fast activity; and (iii) seizure termination: including the last 10 s of the seizure discharge. For each of these periods, we calculated the h2 coefficient, a non-linear correlation coefficient that evaluates the functional connectivity across two regions.

Electrical Stimulation: Stimulation was applied to the thalamus at frequencies varying from 1 to 200 Hz. Pulse width was set to 100 μs and amplitude was set to 1-5 mA. The behavior of epileptogenic discharges (i.e., interictal spikes) in the epileptogenic zone was monitored. Specifically we evaluated the rate of interictal spikes and their amplitude in three time windows: pre stimulation (30 seconds before the stimulations was applied), stimulation (while stimulation was applied) and post stimulation (considering the 30 seconds after stimulation was interrupted). We used bootstrap to assess statistical significance among these intervals.

### Results

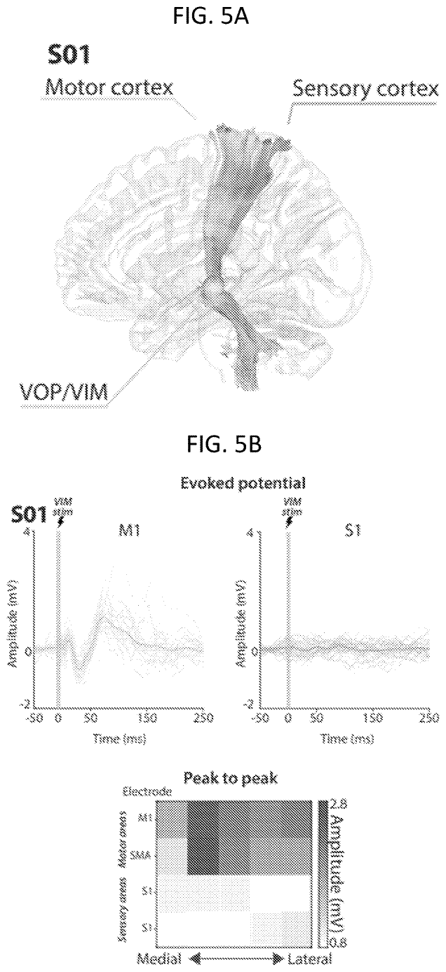

We verified the thalamocortical pathways by means of electrophysiology and imaging techniques. Tractography revealed the presence of fibers projecting from the motor thalamus towards the motor cortex (FIG. 5). Following low-frequency stimulation, we identified clear evoked potentials in ipsilateral M1, reflecting orthodromic monosynaptic neurotransmission (FIG. 5).

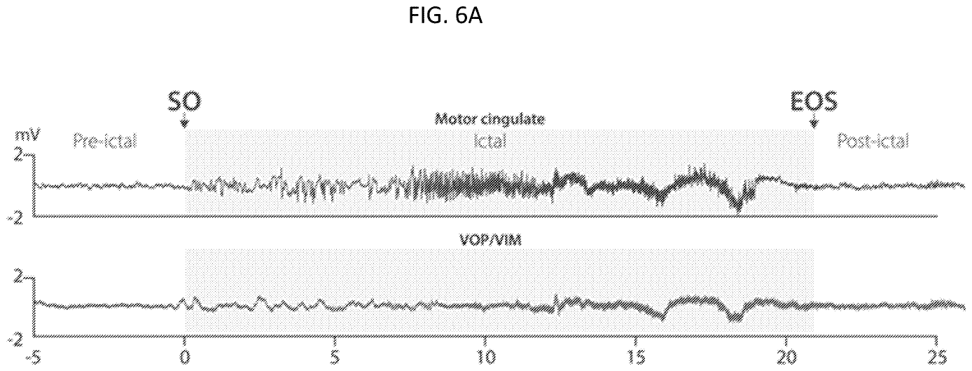

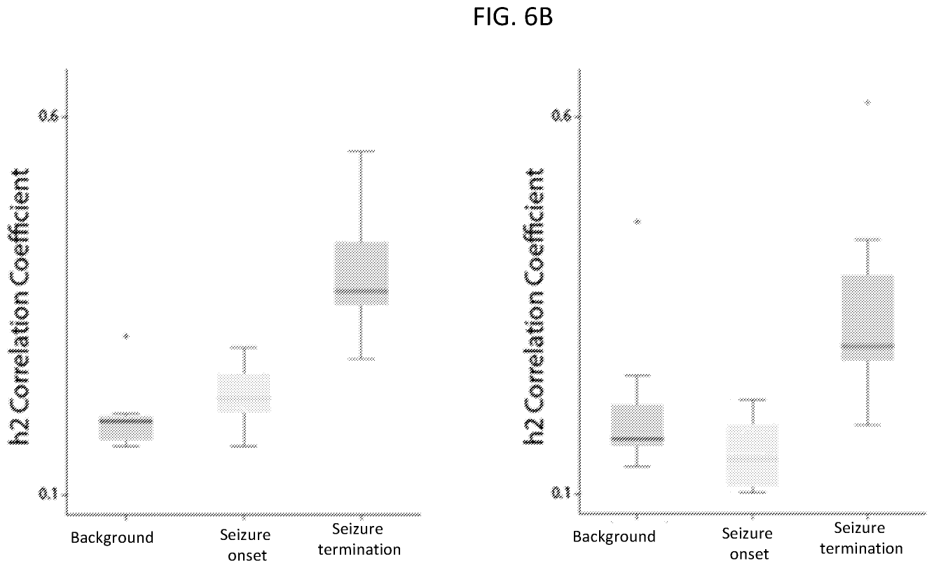

Our connectivity analysis revealed an involvement of the motor thalamus observed during the course of the 59 analyzed seizures. As depicted in FIG. 6, the correlation between the motor thalamus and the epileptogenic zone (moto cingulate) was maximal around seizure termination. These results suggest that a thalamo-cortical coupling could appear early after the onset of focal Rolandic seizures and then sustainably increase until raising its maximal level at seizure termination.

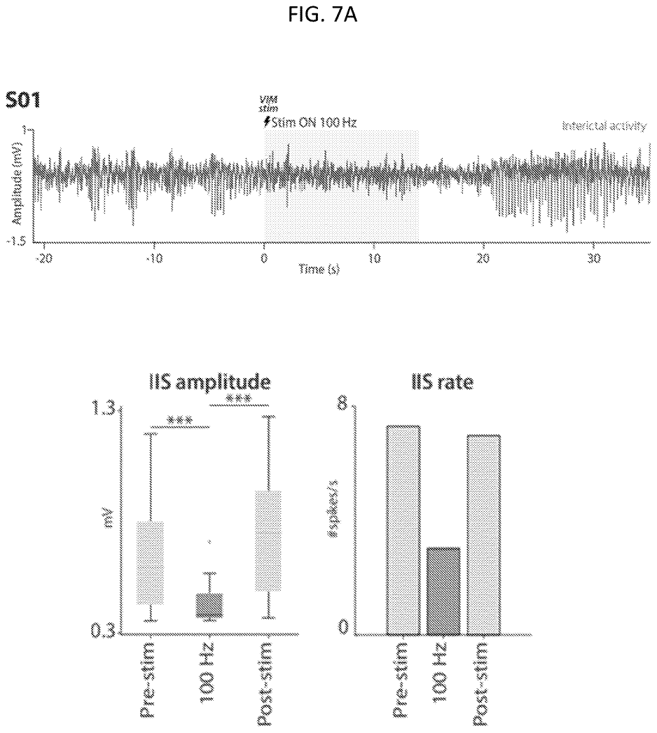

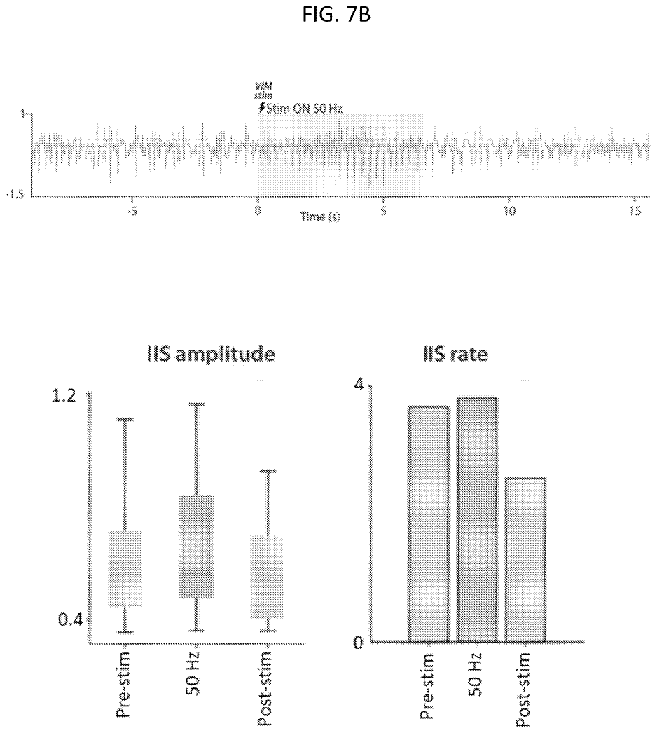

Electrical stimulation of the VIM nucleus of the motor thalamus affected the epileptogenic zone in a frequency-dependent manner. As shown in FIG. 7, we observed that frequency stimulation of 100 Hz significantly decreased both the rate and amplitude of pathological interictal spikes.

It will be apparent that the precise details of the methods or compositions described may be varied or modified without departing from the spirit of the described implementations. We claim all such modifications and variations that fall within the scope and spirit of the claims below.

1. A method for treating focal epilepsy in a subject, comprising:
applying a therapeutically effective amount of an electrical stimulus to thalamic neurons, or to sub-thalamic white matter fibers that project to the thalamic neurons, with one or more electrodes of a deep brain stimulator implanted in the thalamus or subthalamus of the subject and controlled by a neurostimulator, and wherein:
the thalamic neurons project to a target location in the premotor, motor, or sensory cortex of the subject;
the target location comprises an epileptogenic zone for seizure-induced motor or sensory impairment due to the focal epilepsy in the subject; and
the electrical stimulus is applied in an amount sufficient to reduce the seizure-induced motor or sensory impairment in the subject.

2. The method of claim 1, wherein the focal epilepsy is medically refractory focal epilepsy.

3. The method of claim 1, wherein applying the therapeutically effective amount of the electrical stimulus inhibits epileptiform activity in the epileptogenic zone.

4. The method of claim 1, wherein the electrical stimulus comprises electrical pulses having a pulse frequency of between about 50 Hz and about 1000 Hz.

5. The method of claim 1, wherein the electrical stimulus comprises electrical pulses having an amplitude of less than about 10 mA, pulse widths between about 100 μs and about 2 ms, and/or a pulse frequency between about 100 Hz and about 1000 Hz.

6. The method of claim 1, wherein the electrical stimulus comprises electrical pulses having a pulse frequency of between about 100 Hz and about 250 Hz.

7. The method of claim 1, wherein the electrical stimulus comprises electrical pulses having a pulse frequency of between about 150 Hz and about 200 Hz.

8. The method of claim 1, wherein the electrical stimulus comprises electrical pulses having a pulse frequency of about 100 Hz.

9. The method of claim 1, wherein the electrical stimulus is applied to neurons in at least one motor and/or sensory area(s) of the ventral thalamus.

10. The method of claim 9, wherein the electrical stimulus is applied to the ventral anterior nucleus (VA), the ventral oralis posterior nucleus (VOP), the ventral oralis anterior nucleus (VOA), the ventralis intermediate nucleus (VIM), the ventralis caudalis (VC).

11. The method of claim 10, wherein the electrical stimulus is applied to the ventralis intermediate nucleus (VIM).

12. The method of claim 10, wherein the electrical stimulus is applied to the ventral oralis posterior nucleus (VOP).

13. The method of claim 1, wherein the thalamic neurons are located with a region of the thalamus defined by the following AC/PC stereotactic coordinates: Anterior-Posterior: 4-10 mm from PC; Mesial-Lateral: 12-16 mm from the AC/PC line; and Dorsal-Ventral: +2 to −6 mm from the AC/PC plane.

14. The method of claim 1, wherein the electrical stimulus is applied to the white matter fibers in the Field H₁ of Forel of the sub-thalamic area.

15. The method of claim 1, wherein the neurostimulator is activated to apply the electrical stimulus in response to feedback from one or more electrodes for sensing neural activity at the target location in the subject.

16. The method of claim 15, wherein the feedback is epileptiform activity indicating the presence or onset of a seizure in the subject.

17. The method of claim 1, wherein the neurostimulator is configured for activation by the subject in response to sensation of an aura of the focal epilepsy.

18. The method of claim 1, wherein the neurostimulator is an external or implanted pulse generator.

19. The method of claim 1, wherein the neurostimulator delivers the electrical stimulus as charge balanced pulses, a continuous electrical stimulus, and/or a closed-loop electrical stimulus.

20. The method of claim 1, wherein the target location is an epileptogenic zone for seizure-induced motor impairment in the subject and the electrical stimulus is applied at or below a motor threshold such that the electrical stimulus does not directly elicit movement in the subject.

21. The method of claim 1, wherein:
the focal epilepsy is Rolandic epilepsy, the electrodes of the deep brain stimulator are implanted at the motor thalamus, and the electrical stimulus comprises electrical pulses having a pulse frequency of between 100 Hz and 250 Hz.

22. The method of claim 1, further comprising selecting the subject with the focal epilepsy for treatment and implanting the deep brain stimulator in the subject.

23. The method of claim 1, wherein the electrical stimulus is not applied to the anterior or centromedian nucleus of the thalamus.
